(19) United States
(12) Patent Application Publication
(10) Pub. No.: US 2006/0129058 A1
(43) Pub. Date: Jun. 15, 2006

(54) Electromyogram for animals

(75) Inventors: James Strong, Indiana, PA (US); George W. Power, Clarksburg, PA (US); Errin Miller, Blairsville, PA (US)

Correspondence Address: KIRKPATRICK & LOCKHART NICHOLSON GRAHAM LLP, 535 SMITHFIELD STREET, PITTSBURGH, PA, 15222, US

(73) Assignee: Power Equine

(21) Appl. No.: 11/010,783

(22) Filed: Dec. 13, 2004

(51) Int. Cl. A61B 5/04

(52) U.S. Cl. 600/546; 128/903

(57) ABSTRACT

A system for receiving at least one electromyogram signal representing physiological activity in a quadruped. The system may comprise a first probe and a wireless receiver in wireless communication with the first probe. Also, an electromyogram device for determining a neuromuscular balance of a quadruped. The device may comprise a first probe and a second probe. The device may also comprise a processor in communication with the first probe and the second probe. The processor may be configured to compare a first electromyogram signal captured from the first probe and a second electromyogram signal captured from the second probe. Additional embodiments include an electrode for receiving an electromyogram signal representing physiological activity in a quadruped and a method for analyzing the physiology of a quadruped.

### BACKGROUND

Breeders and trainers of animals have a keen interest in determining and improving the health of their animals. Animal performance in even basic physical activities may be affected, for better or worse, by the animal's state of neuromuscular function. The impact of neuromuscular health is even more acute for animals that perform strenuous physical activities. For example, neuromuscular health may affect the performance of a racehorse or race hound, as well as that of a show animal, such as a dog or horse.

Many animal breeders and trainers today spend much money and time to improve the neuromuscular health of their animals. Many regularly hire chiropractors, veterinarians, acupuncturists, massage therapists, and other practitioners to diagnose and correct neuromuscular problems. To date, however, there is no practical objective method to measure the impact that animal chiropractors and other practitioners have on an animal's neuromuscular health.

Human medicine has developed methods for objectively measuring neuromuscular health, and gathering other forms of physiological information, by sensing electrical signals generated by the body such as electromyogram (EMG) signals and electrocardiogram (ECG) signals. For example, in humans, EMG readings are used to measure the intensity of neural signals reaching the muscles. Measuring the intensity of EMG signals as well as comparing EMG signals captured at various parts of the body allows human practitioners to determine neuromuscular balance, an important indicator of neuromuscular health. Also, measuring the intensity of EMG signals at various times, e.g., before and after treatment, provides a comparative picture of neuromuscular health for the patient.

Current devices and methods for measuring bodily electrical signals, such as EMG, are not practical for use on animals for a number of reasons. First, existing methods for EMG require that a series of electrodes make direct and sustained electrical contact with a patient's skin. Many animals have thick coats of fur or hair making it difficult to push electrodes into contact with the animals' skin. Especially since EMG readings are desired from multiple locations along the animal, it may not be practical, or possible, to shave the animal at each of these locations, or to use needle electrodes that cause discomfort to the animal. Also, existing methods for EMG require that electrodes be wired to an external processing device. Wires may tend to make an animal scared or anxious. Also, some animals, such as horses, may not allow themselves to be wired to a stationary object. If they do allow themselves to be attached, it may be difficult to keep an anxious or scared animal still to obtain a good signal or to prevent the animal from ripping off wired electrodes.

Therefore, there is a need for devices, systems and methods for measuring EMG and other bodily electrical signals in animals without shaving the animals or using needle electrodes. There is also a need for devices, systems and methods for measuring EMG and other bodily electrical signals in animals without using wires, and for devices, systems, and methods for evaluating an animal's neuromuscular balance.

### SUMMARY OF THE INVENTION

One embodiment of the present invention relates to a system for receiving at least one electromyogram signal representing physiological activity in a quadruped. The system may comprise a first probe and a wireless receiver in wireless communication with the first probe.

Another embodiment of the present invention relates to an electromyogram device for determining a neuromuscular balance of a quadruped. The device may comprise a first probe and a second probe. The device may also comprise a processor in communication with the first probe and the second probe. The processor may be configured to compare a first electromyogram signal captured from the first probe and a second electromyogram signal captured from the second probe.

Another embodiment of the present invention relates to an electrode for receiving an electromyogram signal representing physiological activity in a quadruped. The electrode may comprise a first surface. The first surface may define at least one groove.

Yet another embodiment of the present invention relates to a method for analyzing the physiology of a quadruped. The method may comprise comparing a first electromyogram signal representing physiological activity on a first side of the quadruped with a second electromyogram signal representing physiological activity on a second side of the quadruped.

The reader will appreciate the foregoing details and advantages of the present invention, as well as others, upon consideration of the following detailed description of embodiments of the invention. The reader also may comprehend such additional details and advantages of the present invention upon making and/or using embodiments within the present invention.

### BRIEF DESCRIPTION OF THE FIGURES

FIG. 1 is a block diagram of a system according to various embodiments;

FIG. 2 is a block diagram of a probe assembly according to various embodiments;

FIG. 3A is a side view of a probe assembly according to various embodiments;

FIG. 3B is a bottom view of a probe assembly according to various embodiments;

FIG. 4A is a bottom view of an electrode according to various embodiments;

FIG. 4B is a side view of an electrode according to various embodiments;

FIG. 5 is a block diagram of a system according to various embodiments;

FIG. 6 is a flowchart of a process flow according to various embodiments; and

FIG. 7 is a diagram of an animal according to various embodiments.

### DESCRIPTION

It is to be understood that the figures and descriptions of the present invention have been simplified to illustrate elements that are relevant for a clear understanding of the present invention, while eliminating, for purposes of clarity, other elements. Those of ordinary skill in the art will recognize, however, that these and other elements may be desirable. However, because such elements are well known in the art, and because they do not facilitate a better understanding of the present invention, a discussion of such elements is not provided herein.

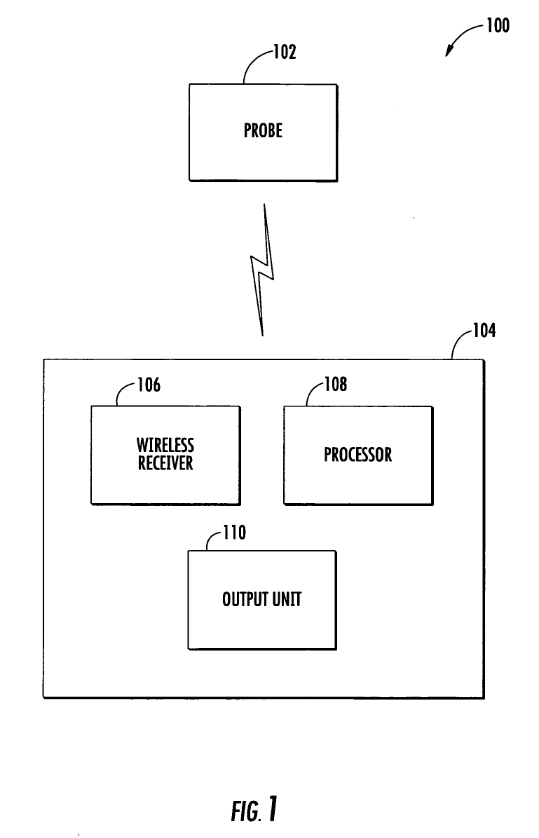

FIG. 1 shows a functional block diagram of a system 100 for capturing and processing one or more bodily electrical signals from an animal. The electrical signals may be any kind of bodily electrical signals representing physiological activity in the animal including, for example, electromyogram (EMG) signals, electrocardiogram (ECG) signals, etc. The system 100 may be adapted to process electrical signals from any kind of animal. For example, the system 100 may be adapted to process electrical signals from a quadruped such as a horse or dog.

The system 100 may comprise a probe 102 and a base unit 104. The base unit 104 may include a wireless receiver 106, a processor 108 and an output unit 110. In various embodiments, the system 100 may include more than one probe 102 as discussed in more detail below.

The probe 102 may be adapted to capture an EMG or other bodily electrical signal from the animal and wirelessly transmit the signal to the base unit 104 for further processing. The wireless receiver 106 of the base unit 104 may receive one or more EMG signals transmitted from the probe 102. The processor 108 of the base unit 104 may be any suitable microprocessor and may include peripheral components such as, for example, memory and data storage. In various embodiments, the processor 108 may evaluate EMG signals received from the probe 102. For example, the processor 108 may compare two or more EMG signals and/or perform various filtering, and scaling of EMG signals as required. The output unit 110 may be any type of device suitable for printing or displaying EMG signals or analysis thereof. For example, output unit 110 may be a computer screen, printer, etc. In various embodiments, the output unit 110 may be a portable device such as a Personal Digital Assistant (PDA), other handheld device, or laptop computer.

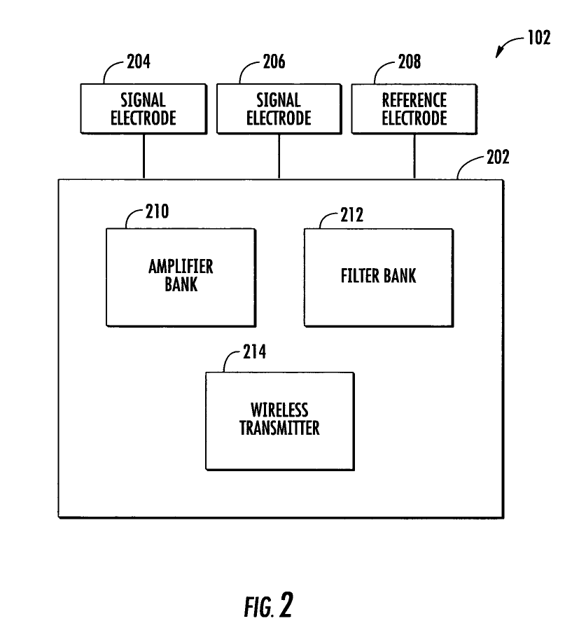

FIG. 2 shows a functional block diagram of a probe 102 for use with the system 100 according to various embodiments. Probe 102 may include signal electrodes 204, 206, reference electrode 208 and signal processing unit 202. The signal processing unit 202 may include amplifier bank 210, a filter bank 212 and a wireless transmitter 214.

Signal electrodes 204, 206, and reference electrode 208 may be adapted to be placed in contact with the skin of an animal to capture EMG signals according to various embodiments. Each electrode 204, 206, 208 may sense an electric potential on the surface of the animal's skin. This electric potential may correspond to neural activity occurring below the skin. The signal electrodes 204, 206 may be situated across a muscle or nerve of interest, while the reference electrode 208 may be placed at a neutral location on the animal's skin. In various embodiments, the positions of the electrodes 204, 206, 208 relative to each other may be fixed by their connection to the probe 102. The EMG signal may be the difference between the electric potentials at signal electrodes 204, 206 referenced to the potential at reference electrode 208.

The amplifier bank 210 may comprise one or more amplifier stages for receiving EMG signals from electrodes 204, 206, 208 and increasing the intensity of the signals for further processing. The amplifier stages of the amplifier bank 210 may include any suitable analog or digital amplifiers. For example, one or more amplifier stages may utilize an operational amplifier (op-amp) circuit or any other transistor network using any suitable technology, such as, for example, CMOS, or bipolar junction transistors. In various embodiments, one or more amplifier stages of the amplifier bank 210 may be digital amplifiers implemented by a digital signal processor (DSP) or other microprocessor.

The filter bank 212 may comprise one or more filters for filtering frequency components of the EMG signals that do not contain useful information and frequency components that are particularly susceptible to noise. In various embodiments, useful information from an EMG signal may be largely concentrated between 0.05 and 100 Hz. Accordingly, the filter bank 212 may include a band-pass filter, for example, between about 0.05 and about 100 Hz. The filter bank 212 may also include notch filters for removing frequency components in which noise is a particularly prevalent. For example, a notch filter between 50 and 60 Hz may be included to reduce noise due to power-line interference.

The filters of the filter bank 212 may be analog or digital of any suitable order and may be implemented according to any suitable design. For example, the filter bank 212 may include one or more analog filters such as Butterworth filters, Chebyshev filters, Constant K filters, etc. In various other embodiments, a digital signal processor (DSP) may implement one or more filters of filter bank 212. The DSP may include peripherals including memory, data storage, etc. Individual filters may be implemented digitally by the DSP according to any suitable digital filter design such as, for example, Butterworth, Chebyshev, Constant K, etc.

The wireless transmitter 214 of the probe 102 may transmit EMG signals to the base unit 104 of the system 100, for example, via the wireless receiver 106. In various embodiments, the wireless transmitter 214 and the wireless receiver 106 may be configured with features of wireless communication that enhance the operation of the system 100. For example, wireless transmitter 214 and receiver 106 may be configured to transmit the EMG signals according to a frequency-hopping scheme, thus providing security and reducing signal degradation due to harmful interference from other radio frequency (RF) sources. Also, the wireless transmitter 214 and receiver 106 may utilize a multiple channel/address scheme allowing the one wireless receiver 106 to receive EMG signals from multiple wireless transmitters 214. For example, the wireless receiver 214 of a first probe 102 may be assigned a first channel or address and wireless receiver of a second probe 102 may be assigned a second channel or address. In various embodiments, these and other features may be utilized by configuring the wireless transmitter 214 and receiver 106 to communicate according to the BLUETOOTH™ standard.

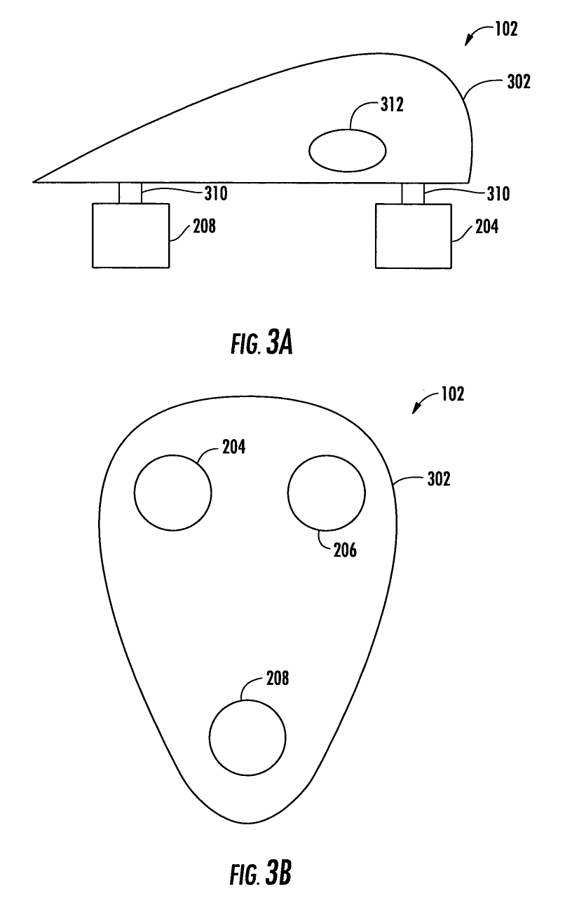

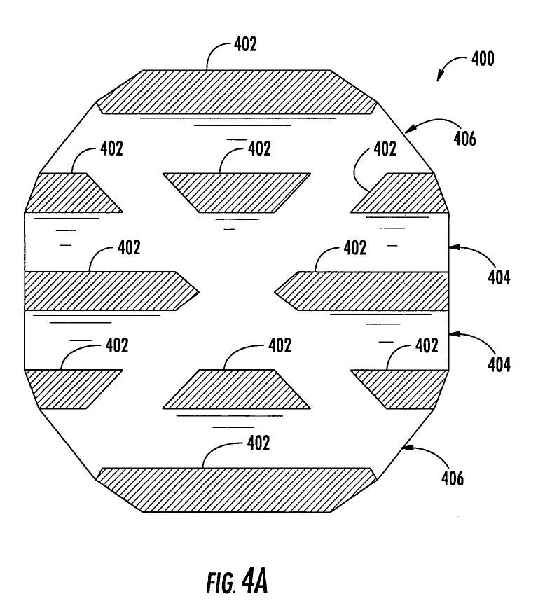

FIGS. 3A and 3B show a physical representation of probe 102 according to various embodiments. Enclosure 302 may house various components of the probe 102 including, for example, signal-processing unit 202. Electrodes 204, 206, 208 may be connected to the probe 102 using post connectors 310, although it is envisioned that any suitable connector may be used. A button 312 may actuate the probe 102 causing it to sense an EMG signal and send the EMG signal to the base unit 104.

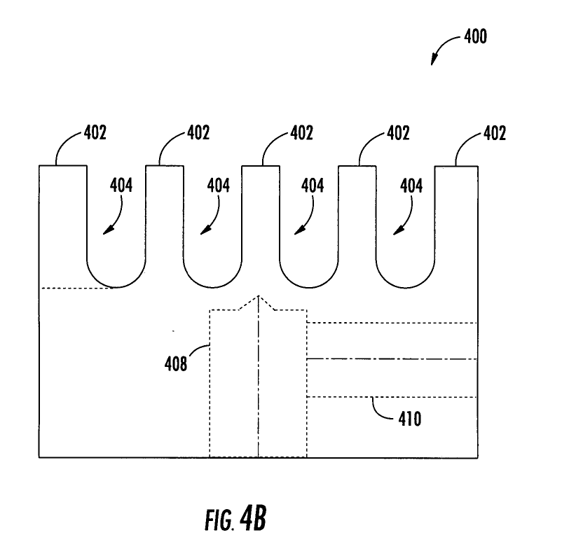

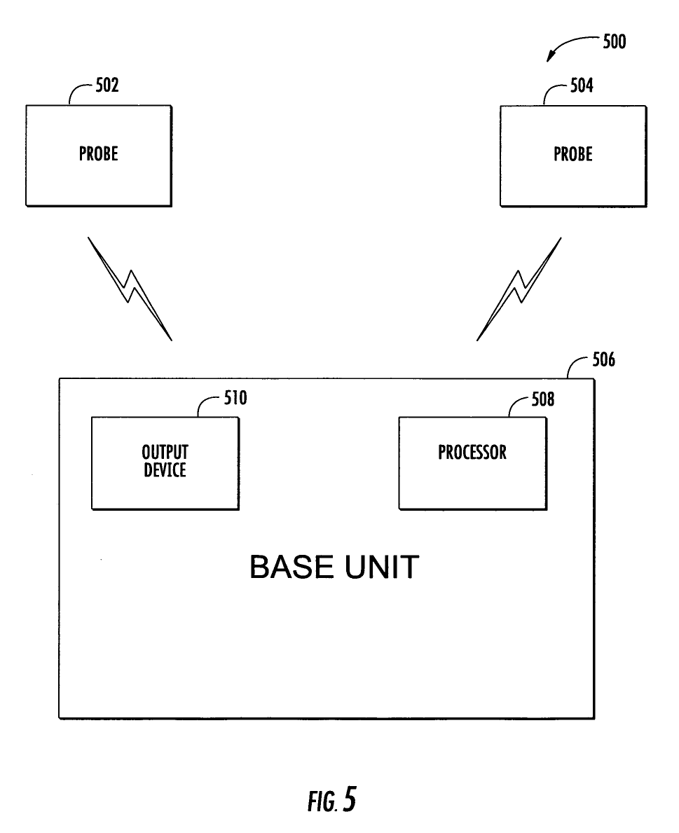

Electrodes 204, 206 and 208, in various embodiments, may be configured to penetrate an animal's hair and sense an EMG signal at the surface of the animal's skin. FIGS. 4A and 4B show views of an exemplary electrode 400 according to various embodiments. It will be appreciated that the exemplary electrode 400 may be used with the system 100 or with any other EMG system. In various embodiments, the electrode 400 may be made of copper, bronze or any other conductor of electrical signals. Electrodes 204, 206, 208 may be substantially shaped as cylinders, polyhedrons, or any other suitable shapes.

The electrode 400 may include at least one protrusion 402 configured to make contact with an animal's skin through its coat. The protrusion or protrusions 402 may create a comb-shaped pattern configured to reach through the animal's hair and may have the effect of guiding the animal's hair into grooves 404, 406 to improve electrical connectivity with the animal's skin. In various embodiments, the surface area of the side of the electrode 400 including protrusions 402, as well as the distance between electrodes 204, 206, 208, may be proportional to the size of the vertebrae of the particular quadruped or other animal whose EMG reading is being captured. Such dimensions are known by those skilled in the art. For example, an electrode 400 for use with a horse may be a substantially cylindrical shape with a diameter of about one inch.

The electrode 400 and the protrusions 402 may define a series of grooves 404, 406. When the electrode 400 is placed in contact with an animal's skin, the grooves 404, 406 may operate to channel the animal's hair allowing protrusions 402 to contact the animal's skin. The grooves 404, 406 may be arranged in various patterns to maximize their efficiency in channeling the animal's hair. For example, some of the grooves 404 may be substantially parallel to one another. Other grooves 406 may not be parallel to one another and may intersect one another 406 as well as grooves 404. Such configurations may allow the practitioner to move the electrodes along the animal to various locations while maintaining electrical contact with the animal's skin. This may allow greater simplicity than conventional electrodes.

In various embodiments, electrode 400 may also include a connector 408 adapted to connect to a corresponding connector, such as post connector 310 of probe 102. The connector 408 may be any suitable connector known in the art, such as a male or female connector. In various embodiments, the connector 408 may take the form of a hole for receiving a post connector. The electrode 400 may also include a setscrew hole 410 for receiving a setscrew (not shown), which may secure the electrode 400 to a post connector.

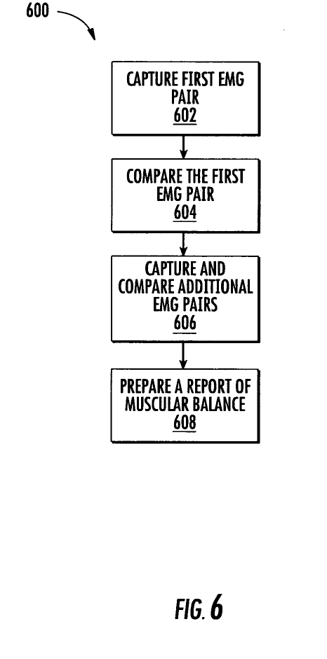

FIG. 5 shows a functional block diagram of a system 500 for analyzing bodily signals from an animal according to various embodiments. The system 500 may include one or more probes 502, 504 and a base unit 506. The probes 502, 504 may be similar in structure to the probe 102 described above. Probe 502 and probe 504 may be adapted to sense EMG signals in a spinal region of the animal, in certain applications, on opposite sides of the animal's body. The probes 502, 504 may sense their respective EMG signals at substantially the same time as discussed in more detail below.

The probes 502, 504 may transmit sensed EMG or other bodily electrical signals to the base unit 506. In various embodiments, the probes 502, 504 may transmit to the base unit 506 wirelessly, for example, as described above with respect to FIG. 1. The probes 502, 504 may also transmit to the base unit 506 via a wired connection. Wired communication between probes 502, 504 and base unit 506 may be in analog or digital and may be formatted according to any serial or parallel standard including, for example, the Universal Serial Bus digital standard, the IEEE 1394 digital standard, etc.

The base unit 506 may include various components including an output unit 510 and a processor 508. The output unit 510, in various embodiments, may be similar to the output unit 110 described above with reference to FIG. 1. The processor 508 may also be similar to processor 108 described above. Also, the processor 508 may find a neuromuscular balance reading for the animal by comparing an EMG reading taken on one side of the animal with a corresponding neuromuscular reading on an opposite side of the animal.

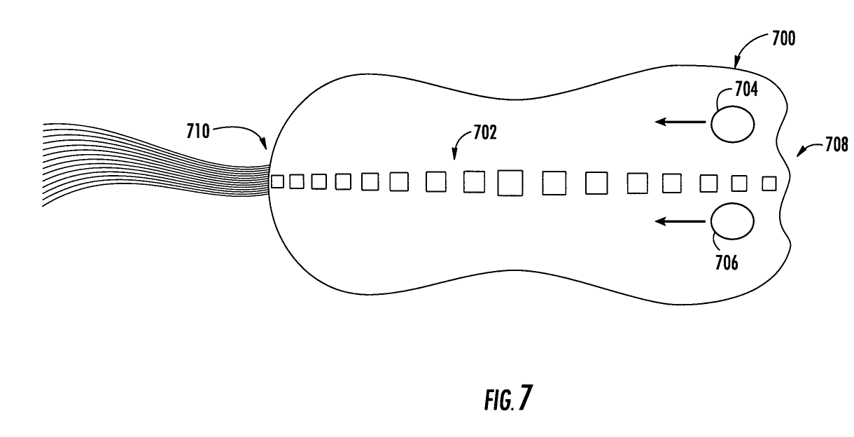

FIG. 6 is a flowchart of a process flow 600 for capturing and processing an EMG signal from an animal according to various embodiments. At step 602, a first EMG measurement pair including first and second EMG signals may be captured from the animal. The first and second EMG signals may be captured in the same spinal region of the animal, albeit on opposite sides. For example, the first EMG signal may be captured in the thoracic region on the animal's right side, while the second EMG signal may be captured in the thoracic region but on the animal's left side.

FIG. 7 is a diagram of an exemplary animal 700 having an EMG reading taking according to process flow 600. Reference number 708 points to the cervical region and reference number 710 points to the sacral region of the animal's spine 702. The first and second EMG signals of the first EMG measurement pair may be captured by probes 704 and 706. In various embodiments the first and second EMG signals may be captured close enough in time that they both correspond to at least one common physiological event. Accordingly, the first and second EMG signals may represent corresponding muscle activity on the right and left side of the animal in response to the same impulse from the animal's brain.

Referring back to FIG. 6, at step 604, the first EMG measurement pair may be compared, for example, to determine symmetry in intensity. Symmetry, or lack thereof, in the intensities of the first and second EMG signals may be an indication of the neuromuscular balance of the animal 700. A lack of symmetry may indicate a neuromuscular problem or defect in the animal 700.

At step 606, additional EMG measurement pairs from other portions of the animal's body may be captured and compared. The additional EMG measurement pairs may include pairs captured at different locations in the same spinal region as the first EMG measurement pairs as well as pairs captured in other spinal regions. In various embodiments, the EMG measurement pairs may be captured sequentially along the animal's spine 702. For example, the first EMG measurement pair may be captured in the animal's cervical region 708. A subsequent EMG measurement pair may be captured in the thoracic region with further EMG measurement pairs captured in turn in the lumbar and then sacral regions 710. It will be appreciated that EMG measurement pairs could also be taken in the opposite direction, e.g., from the sacral region 710 to the cervical region 708.

At step 608, a report of the animal's neuromuscular balance may be prepared. The report may detail the symmetry, or lack thereof, between the EMG signals at various points in the animal's 700 various spinal regions. It will be appreciated that the report may be used to determine the animal's 700 aptitude for a particular task, e.g. horse racing. It will also be appreciated that the report may be used to develop a line of treatment, such as chiropractic treatment, to improve the animal's 700 physical performance and/or to access the success of a previous line of treatment.

The benefits of the present apparatus, methods and systems are readily apparent to those skilled in the art. The various embodiments described herein may provide representations of EMG signals or other electrical signals representing physiological activity in the animal. Various portions and components of various embodiments of the present invention, for example digital amplifiers and/or filters, may be implemented in computer software code using any suitable language, for example, Visual Basic, C, C++, or assembly language using, for example, standard or object-oriented techniques.

While several embodiments of the invention have been described, it should be apparent, however, that various modifications, alterations and adaptations to those embodiments may occur to persons skilled in the art with the attainment of some or all of the advantages of the present invention. It is therefore intended to cover all such modifications, alterations and adaptations without departing from the scope and spirit of the present invention as defined by the appended claims.

What is claimed is:

1. A system for receiving at least one electromyogram signal representing physiological activity in a quadruped, the system comprising:
a first probe; and
a wireless receiver in wireless communication with the first probe.

2. The system of claim 1, further comprising a second probe.

3. The system of claim 2, further comprising a processor in communication with the wireless receiver, wherein the processor is configured to compare a first electromyogram signal captured by the first probe and a second electromyogram signal captured by the second probe.

4. The system of claim 1, wherein the first probe comprises a first signal electrode;
and a first wireless transmitter.

5. The system of claim 4, wherein the first probe further comprises:
an amplifier in communication with the first signal electrode and the first wireless transmitter; and
a digital signal processor in communication with the first signal electrode and the first wireless transmitter.

6. The system of claim 5, wherein the digital signal processor is configured to filter noise from the first electromyogram signal.

7. The system of claim 4, wherein the first probe further comprises:
a second signal electrode; and
a reference electrode.

8. The system of claim 7, wherein the first signal electrode, the second signal electrode and the reference electrode are arranged in a triangular configuration.

9. The system of claim 7, wherein the first signal electrode, the second signal electrode and the reference electrode are configured to obtain an electromyogram signal from a quadruped.

10. The system of claim 4, wherein the first signal electrode comprises a first surface defining at least one groove.

11. The system of claim 10, wherein the first surface defines a plurality of grooves

12. The system of claim 11, wherein the at least a portion of the plurality of grooves are substantially parallel to one another.

13. The system of claim 11, wherein at least one of the plurality of grooves is not substantially parallel to another of the plurality of grooves.

14. The system of claim 11, wherein at least one of the plurality of grooves intersects at least one other of the plurality of grooves on the first side of the first signal electrode.

15. The system of claim 10, wherein the first signal electrode is a substantially cylindrical shape, and wherein the first surface of the first signal electrode is on one end of the substantially cylindrical shape.

16. The system of claim 1, wherein the first probe further comprises a switch configured to activate a first wireless transmitter.

17. The system of claim 1, wherein the wireless receiver is configured to receive wireless transmissions from devices at a plurality of addresses over a single channel.

18. The system of claim 17, further comprising a second probe comprising a second wireless transmitter, wherein the first probe comprises a first wireless transmitter, and wherein a first address of the plurality of addresses is assigned to the first wireless transmitter and a second address of the plurality of addresses is assigned to the second wireless transmitter.

19. The system of claim 1, wherein the wireless receiver and a first wireless transmitter included in the first probe are configured to communicate according to a frequency-hopping scheme.

20. An electromyogram device for determining a neuromuscular balance of a quadruped, the device comprising:
a first probe;
a second probe;
a processor in communication with the first probe and the second probe, wherein the processor is configured to compare a first electromyogram signal captured from the first probe and a second electromyogram signal captured from the second probe.

21. The device of claim 20, wherein the first probe comprises:
a first signal electrode;
a second signal electrode; and
a reference electrode.

22. The device of claim 21, wherein the first signal electrode, the second signal electrode and the reference electrode are arranged in a triangular configuration.

23. The device of claim 21, wherein the first signal electrode, the second signal electrode and the reference electrode are configured to obtain an electromyogram signal from a quadruped.

24. The device of claim 20, wherein the first probe comprises a first signal electrode, and wherein the first signal electrode comprises a first surface defining at least one groove.

25. The device of claim 24, wherein the first surface defines a plurality of grooves and wherein at least a portion of the plurality of grooves are substantially parallel to one another.

26. The device of claim 24, wherein the first surface defines a plurality of grooves, and wherein at least one of the plurality of grooves is not substantially parallel to another of the plurality of grooves.

27. The device of claim 24, wherein the first surface defines a plurality of grooves, and wherein at least one of the plurality of grooves intersects at least one other of the plurality of grooves on the first side of the electrode.

28. The device of claim 24, wherein the first signal electrode is a substantially cylindrical shape, and wherein the first surface is one end of the substantially cylindrical shape.

29. An electrode for receiving an electromyogram signal representing physiological activity in a quadruped, the electrode comprising:
a first surface; and
wherein the first surface defines at least one groove.

30. The electrode of claim 29, wherein at least a portion of the first signal electrode comprises an electrically conductive material.

31. The electrode of claim 29, wherein the electrode is shaped as a cylinder, and wherein the first surface of the electrode corresponds to a first flat side of the cylinder.

32. The electrode of claim 31, wherein the electrode is about one inch in diameter.

33. The electrode of claim 29, wherein the electrode is shaped as a polyhedron, and wherein the first surface of the electrode corresponds to a first flat side of the polyhedron.

34. The electrode of claim 29, wherein a second surface of the electrode comprises an electrical connector.

35. The electrode of claim 29, wherein the first surface of the electrode is proportional to the vertebrae size of the quadruped.

36. The electrode of claim 29, wherein the first surface of the electrode defines a plurality of grooves.

37. The electrode of claim 36, wherein the plurality of grooves are arranged in a comb-shaped pattern.

38. The electrode of claim 37, wherein at least a portion of the plurality of grooves are substantially parallel to one another.

39. The electrode of claim 37, wherein at least one of the plurality of grooves is not substantially parallel to another of the plurality of grooves.

40. The electrode of claim 37, wherein at least one of the plurality of grooves intersects at least one other of the plurality of grooves on the first surface of the electrode.

41. The electrode of claim 29, wherein the electrode is a substantially cylindrical shape, and wherein the first surface of the electrode is on one end of the substantially cylindrical shape.

42. A method for analyzing the physiology of a quadruped, the method comprising:
comparing a first electromyogram signal representing physiological activity on a first side of the quadruped with a second electromyogram signal representing physiological activity on a second side of the quadruped.

43. The method of claim 42, further comprising:
receiving the first electromyogram signal from a first probe; and
receiving the second electromyogram signal from a second probe.

44. The method of claim 42, further comprising
wirelessly transmitting the first electromyogram signal from a first site on the first side of the quadruped to a processing device; and
wirelessly transmitting the second electromyogram signal from a second site on the second side of the quadruped to the processing device.

45. The method of claim 44, wherein the first electromyogram signal and the second electromyogram signal are transmitted to the processing device utilizing a single communications channel.

46. The method of claim 42, wherein the first side of the animal is at least one of a right side and a left side of the animal and the second side is opposite the first side.

47. The method of claim 42, wherein the first electromyogram signal represents physiological activity relating to a first physiological function on the first side of the animal's body and the second electromyogram signal represents physiological activity relating to a corresponding physiological function on the second side of the animal's body.

48. The method of claim 42, wherein the animal is at least one of a horse and a dog.
